(19) United States
(12) Patent Application Publication
(10) Pub. No.: US 2022/0183634 A1
(43) Pub. Date: Jun. 16, 2022

(54) INSPECTION VALUE PREDICTION DEVICE, INSPECTION VALUE PREDICTION SYSTEM, AND INSPECTION VALUE PREDICTION METHOD

(71) Applicants: Kabushiki Kaisha Toshiba, Tokyo (JP); Toshiba Digital Solutions Corporation, Kawasaki-shi (JP)

(72) Inventors: Shintarou SUGIMOTO, Akiruno (JP); Toshinori SAKUMA, Kawasaki (JP); Shingo MIYAZAKI, Fuchu (JP); Hidekazu KURITA, Fuchu (JP)

(73) Assignees: Kabushiki Kaisha Toshiba, Tokyo (JP); Toshiba Digital Solutions Corporation, Kawasaki-shi (JP)

(21) Appl. No.: 17/652,946

(22) Filed: Mar. 1, 2022

Related U.S. Application Data
(63) Continuation of application No. PCT/JP2020/030307, filed on Aug. 7, 2020.

(30) Foreign Application Priority Data
Sep. 25, 2019 (JP) 2019-173968

(51) Int. Cl. A61B 5/00; G16H 50/20; G06N 3/08

(52) U.S. Cl. CPC A61B 5/7275; A61B 5/7264; G06N 3/08; G16H 50/20

(57) ABSTRACT

According to one embodiment, an inspection value prediction device includes a prediction unit and an inverse prediction unit. The prediction unit inputs an inspection value of a user to a first learned model, which has been learned using a first learning data set containing series data of a plurality of inspection values of the user, to predict a future inspection value of the user. When a target value pertaining to the future inspection value of the user is designated, the inverse prediction unit performs an inverse prediction of the inspection value that causes the designated target value to be output when the inspection value is input to the first learned model.

### CROSS-REFERENCE TO RELATED APPLICATION(S)

This application is based upon and claims the benefit of priority from PCT Application No. PCT/JP2020/030307 filed on Aug. 7, 2020, which is based upon and claims the benefit of priority from Japanese Patent Application No. 2019-173968 filed in Japan on Sep. 25, 2019; the entire contents of (all of) which are incorporated herein by reference.

### FIELD

Embodiments described herein relate generally to an inspection value prediction device, an inspection value prediction system, and an inspection value prediction method.

### BACKGROUND

Conventionally, an art is known in which a future inspection value of a user is predicted by generating a prediction model of inspection values using time-series data of inspection values of individual health diagnosis as learning data and inputting the inspection values of a user's health diagnosis into the generated prediction model.

However, the conventional technology may not be sufficient to provide information for improving the lifestyle of the user.

### BRIEF DESCRIPTION OF THE DRAWINGS

FIG. 1 shows an example of an inspection value prediction device according to a first embodiment.

FIG. 2 shows an example of the configuration of a prediction unit and an inverse prediction unit.

FIG. 3 shows an example of contents of a first data table.

FIG. 4 shows a prediction process.

FIG. 5 shows an example of contents of a second data table.

FIG. 6 shows an example of contents of a charge management table.

FIG. 7 shows an inverse prediction process.

FIG. 8 shows an example of the inverse prediction process on an improvement inspection value.

FIG. 9 shows another example of the inverse prediction process on the improvement inspection value.

FIG. 10 shows an example of a list display of an improvement inspection value.

FIG. 11 shows an example of a ranking display of the improvement inspection value.

FIG. 12 shows an example of an inspection value prediction process.

FIG. 13 shows an example of an inspection value inverse prediction process.

FIG. 14 shows an operation of a server device.

FIG. 15 shows an example of the inverse prediction process on the improvement inspection value.

FIG. 16 shows an example of the inspection value inverse prediction process.

### DETAILED DESCRIPTION

According to one embodiment, an inspection value prediction device includes a prediction unit and an inverse prediction unit. The prediction unit inputs an inspection value of a user to a first learned model, which has been learned using a first learning data set containing series data of a plurality of inspection values of the user, to predict a future inspection value of the user. When a target value pertaining to the future inspection value of the user is designated, the inverse prediction unit performs an inverse prediction of the inspection value that causes the designated target value to be output when the inspection value is input to the first learned model.

Hereinafter, an inspection value prediction device, an inspection value prediction system, and an inspection value prediction method according to embodiments will be described with reference to the drawings.

The inspection value prediction device is realized by one or more processors. The inspection value prediction device predicts the future inspection value of the user by inputting the inspection value of the health diagnosis received by the prediction target user into the prediction model. The prediction model is, for example, a model learned using a first learning data set including series data of a plurality of inspection values of the same user. In addition, the inspection value prediction device predicts how the future inspection value of the user will change when the value obtained by improving the inspection value of the health diagnosis received by the prediction target user is input to the prediction model. In addition, the inspection value prediction device performs inverse prediction of what kind of inspection value should be input to the prediction model to output the designated target value when the target value for the future inspection value is designated by the prediction target user. The various inspection values described below are for one or more inspection items.

### First Embodiment

### [Overall Structure]

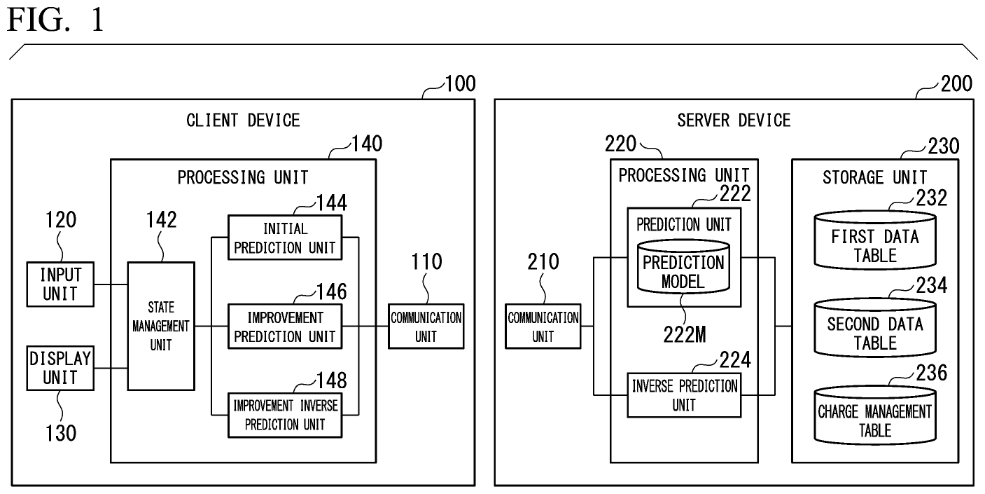

FIG. 1 is a configuration diagram centered on a server device 200, which is an example of the inspection value prediction device of the first embodiment. When the prediction target user inputs an instruction to start the inspection value prediction process to a client device 100, the server device 200 receives the instruction from the client device 100 and starts the prediction process of the inspection value of the prediction target user. Further, when the prediction target user inputs an instruction to start the inverse prediction process of the inspection value to the client device 100, the server device 200 receives the instruction from the client device 100 and starts the inverse prediction process of the inspection value of the prediction target user.

### [Client Device]

The client device 100 is, for example, a terminal device owned by the prediction target user. The client device 100 includes, for example, a communication unit 110, an input unit 120, a display unit 130, and a processing unit 140.

The communication unit 110 includes a communication interface such as a NIC (Network Interface Card) or a wireless communication module. The communication unit 110 communicates with the server device 200 via a network. The network includes the Internet, a communication line, a cellular network, a Wi-Fi network, and the like.

The input unit 120 is a user interface such as a button, a keyboard, and a mouse. The input unit 120 accepts the operation of the prediction target user. The operation of the prediction target user includes, for example, an operation of inputting a current inspection value of the prediction target user and an operation of designating a prediction type to be used when predicting a future inspection value of the prediction target user. The input unit 120 may be a touch panel configured integrally with the display unit 130.

The display unit 130 includes a display device such as an LCD (Liquid Crystal Display) or an organic EL display. The display unit 130 displays, for example, an image showing the prediction result of the inspection value of the prediction target user executed by the server device 200.

The processing unit 140 includes, for example, a state management unit 142, an initial prediction unit 144, an improvement prediction unit 146, and an improvement inverse prediction unit 148. These components are realized by, for example, a hardware processor such as a CPU executing a program (software). The program is an application program provided by a browser, the server device 200, or the like, or a script attached to a web page downloaded by the browser. Some or all of these components may be realized by hardware such as LSI, ASIC, FPGA, GPU (circuit part; including circuitry), or may be realized by the cooperation of software and hardware.

The state management unit 142 manages the type of prediction of the inspection value designated by the prediction target user through the input unit 120. Types of predictions include, for example, initial predictions, improvement predictions, and inverse improvement predictions. The initial prediction is a prediction of future inspection values performed by the prediction target user at the first stage. The improvement prediction is a prediction of future inspection values based on the premise that the current inspection values have improved. The improvement inverse prediction is a prediction of the current inspection value required to match the future inspection value with the target value.

When the type of prediction designated by the prediction target user through the input unit 120 is the initial prediction, the initial prediction unit 144 transmits information, which includes the inspection value of the prediction target user input to the input unit 120 and is for requesting the initial prediction based on the instruction from the state management unit 142, to the server device 200 by using the communication unit 110.

When the type of prediction designated by the prediction target user through the input unit 120 is the improvement prediction, the improvement prediction unit 146 transmits information, which includes the inspection value of the prediction target user input to the input unit 120 and is for requesting improvement prediction based on the instruction from the state management unit 142, to the server device 200 by using the communication unit 110.

When the type of prediction designated by the prediction target user through the input unit 120 is the improvement inverse prediction, the improvement inverse prediction unit 148 transmits information, which includes the inspection value of the prediction target user input to the input unit 120 and is for requesting improvement inverse prediction based on the instruction from the state management unit 142, to the server device 200 by using the communication unit 110.

### [Server Device]

The server device 200 performs a prediction process or an inverse prediction process of the inspection value of the prediction target user, for example, in response to a request from the client device 100. The server device 200 has a function as a web server or an application server. The server device 200 includes, for example, a communication unit 210, a processing unit 220, and a storage unit 230.

The communication unit 210 includes a communication interface such as a NIC (Network Interface Card). The communication unit 210 communicates with the client device 100 via the network. When, for example, the communication unit 210 receives information requesting initial prediction, improvement prediction, or improvement inverse prediction from the client device 100, the communication unit 210 outputs the received information to the processing unit 220.

The processing unit 220 includes, for example, a prediction unit 222 and an inverse prediction unit 224. These components are realized by, for example, a hardware processor such as a CPU executing a program (software). Some or all of these components may be realized by hardware such as LSI, ASIC, FPGA, GPU (circuit part; including circuitry), or may be realized by the cooperation of software and hardware. The program may be stored in advance in a storage device such as an HDD or a flash memory (a storage device including a non-transient storage medium), or may be stored in a removable storage medium (non-transient storage medium) such as a DVD or a CD-ROM, to be installed by attaching the storage medium to a drive device.

When the prediction unit 222 receives information requesting initial prediction or improvement prediction from the client device 100, the prediction unit 222 performs initial prediction or improvement prediction of the inspection value. In this case, the prediction unit 222 predicts the future inspection value of the prediction target user by inputting the inspection value of the prediction target user received from the client device 100 into a prediction model 222M.

When the inverse prediction unit 224 receives the information requesting the improvement inverse prediction from the client device 100, the inverse prediction unit 224 performs the improvement inverse prediction of the inspection value. In this case, the inverse prediction unit 224 performs inverse prediction of what kind of inspection value should be input to the prediction model 222M to output the target value designated by the prediction target user.

The storage unit 230 is realized by, for example, an HDD, a flash memory, a RAM (Random Access Memory), or the like. The storage unit 230 stores, for example, a first data table 232, a second data table 234, and a charge management table 236. The first data table 232 is a data table in which the prediction target user and the inspection value are associated with each other. The second data table 234 is a data table in which the inspection value input to the prediction model 222M and the inspection value output from the prediction model 222M are associated with each other when the prediction unit 222 predicts the future inspection value of the prediction target user. The charge management table 236 is a data table in which the history of the prediction process or the inverse prediction process executed by the prediction target user and a charge content required for the prediction target user for each type of the prediction process are associated with each other.

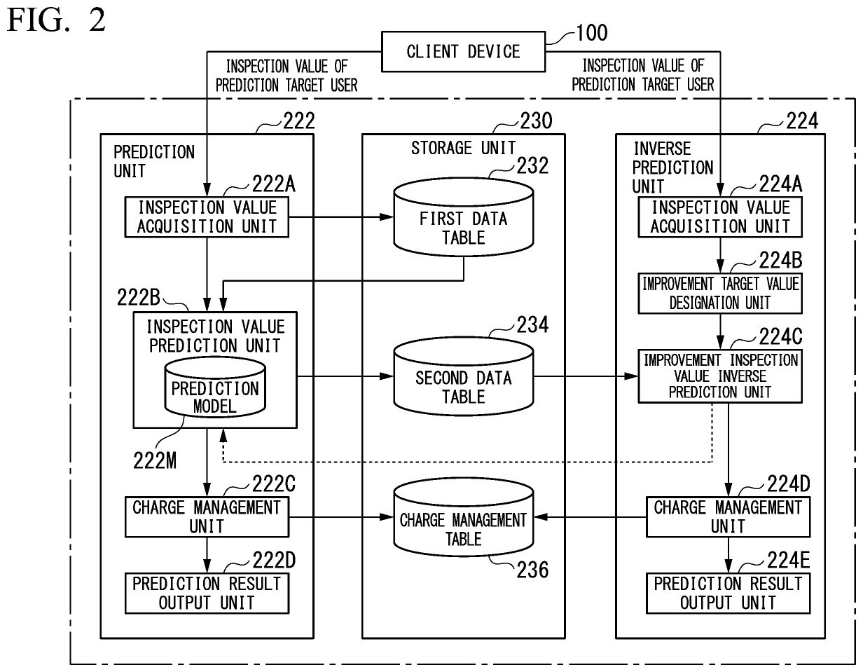

FIG. 2 is a diagram showing an example of the configuration of the prediction unit 222 and the inverse prediction unit 224. The prediction unit 222 includes, for example, an inspection value acquisition unit 222A, an inspection value prediction unit 222B, a charge management unit 222C, and a prediction result output unit 222D.

The inspection value acquisition unit 222A acquires the inspection value input to the client device 100 by the learning target user from the client device 100. The inspection value acquisition unit 222A registers the acquired inspection value in the first data table 232. The inspection value acquisition unit 222A acquires the inspection value from the client device 100, for example, in association with the year in which the learning target user acquired the inspection value, and registers the acquired inspection value in the first data table 232 in association with the user ID of the learning target user. In the present embodiment, the inspection value acquisition unit 222A acquires the inspection value from the client device 100 every year as an example, but may acquire the inspection value from the client apparatus 100 every inspection day.

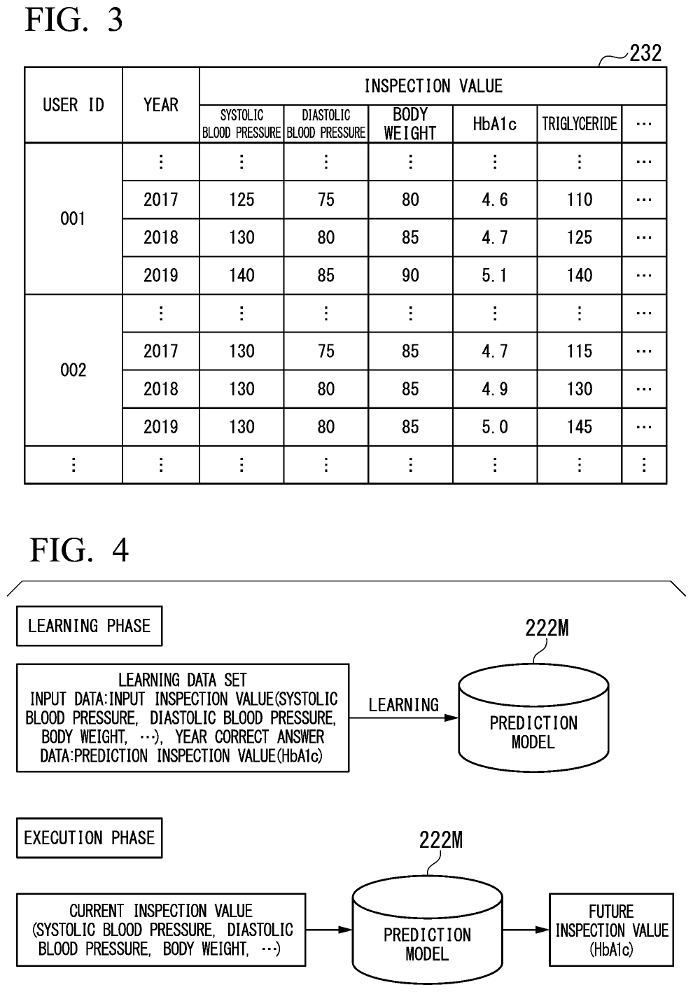

FIG. 3 is a diagram showing an example of the contents of the first data table 232. In the first data table 232, for example, the inspection value for each year is associated with the user ID. In the first data table 232, for example, when a user receives a medical examination a plurality of times in one year, a plurality of inspection values may be associated with one year. The inspection value includes, for example, systolic blood pressure, diastolic blood pressure, body weight, HbA1c, and triglyceride as inspection items. HbA1c is an example of an index for evaluating the disease risk of diabetes, and is an example of the inspection item for predicting an abnormality of a usage prediction user.

The inspection value prediction unit 222B learns the prediction model 222M using the inspection values for each year included in the first data table 232. The prediction model 222M is an example of the “first learned model”. The inspection value prediction unit 222B learns the prediction model 222M by performing machine learning using, for example, a plurality of inspection values associated with the same user ID. The inspection value prediction unit 222B uses, for example, the first inspection value acquired in a relatively early year (first time point) as learning data among the inspection values for each year, and sets it as learning data in a relatively late year (second time point). The prediction model 222M is learned by performing machine learning using the second inspection value acquired at the time point as the correct answer data (teacher data). A data set in which the first inspection value and the second inspection value are associated with each other is an example of the “first learning data set”. The inspection value prediction unit 222B may acquire the prediction model 222M that has been learned by another device. Then, the inspection value prediction unit 222B predicts the future inspection value by inputting the current inspection value acquired by the inspection value acquisition unit 222A into the prediction model 222M.

In the example shown in FIG. 4, the inspection value prediction unit 222B learns the prediction model 222M by performing machine learning using the combination of the input inspection value and the year as the learning data and the prediction inspection value as the correct answer data (teacher data) in the learning phase. The input inspection value includes, for example, systolic blood pressure, diastolic blood pressure, and body weight as inspection items. The prediction inspection value includes, for example, HbA1c as an inspection item.

The inspection value prediction unit 222B predicts future inspection values by inputting the current inspection value acquired by the inspection value acquisition unit 222A into the prediction model 222M in the execution phase. Current inspection values include, for example, systolic blood pressure, diastolic blood pressure, and body weight as inspection items. Future inspection values include, for example, HbA1c as inspection items. The inspection value prediction unit 222B registers the prediction inspection value predicted using the prediction model 222M in the second data table 234 in association with the input inspection value that is the source of the prediction.

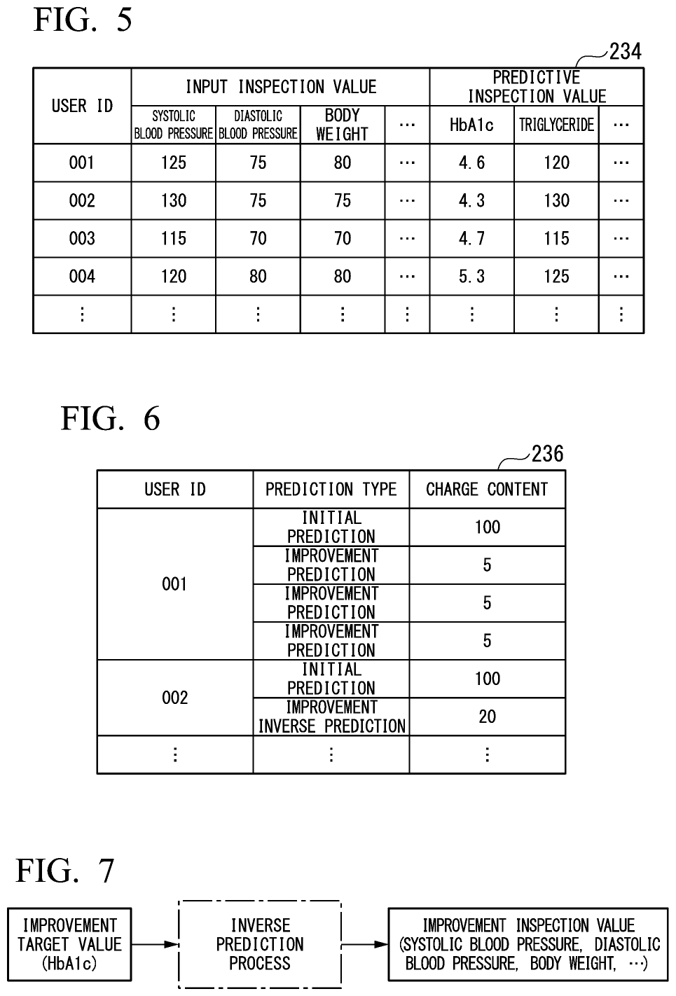

FIG. 5 is a diagram showing an example of the contents of the second data table 234. In the example shown in the figure, the second data table 234 is associated with the input inspection value and the prediction inspection value with respect to the user ID. The input inspection value includes, for example, systolic blood pressure, diastolic blood pressure, and body weight as inspection items. Predictive inspection values include, for example, HbA1c and triglycerides as inspection items.

The charge management unit 222C determines the charge content requested from the prediction target user based on the processing history of the inspection value prediction unit 222B. The charge management unit 222C registers the determined charge content in the charge management table 236 together with the processing history by the inspection value prediction unit 222B.

FIG. 6 is a diagram showing an example of the contents of the charge management table 236. In the illustrated example, in the charge management table 236, the prediction type and the charge content are associated with the user ID. The prediction type includes, for example, a prediction process by the inspection value prediction unit 222B and an inverse prediction process by an improvement inspection value inverse prediction unit 224C described later. In the illustrated example, when the improvement inspection value inverse prediction unit 224C makes an improvement inverse prediction, the charge amount requested from the prediction target user is larger than when the inspection value prediction unit 222B makes the improvement prediction.

The prediction result output unit 222D outputs the future inspection value predicted by the inspection value prediction unit 222B to the client device 100.

The inverse prediction unit 224 includes, for example, an inspection value acquisition unit 224A, an improvement target value designation unit 224B, an improvement inspection value inverse prediction unit 224C, a charge management unit 224D, and a prediction result output unit 224E.

The inspection value acquisition unit 224A acquires the inspection value input to the client device 100 by the learning target user from the client device 100. The inspection value acquisition unit 224A outputs the acquired inspection value to the improvement target value designation unit 224B.

The improvement target value designation unit 224B designates an improvement target value based on the inspection value acquired from the inspection value acquisition unit 224A. The improvement target value designation unit 224B designates, for example, a value obtained by increasing or decreasing the inspection value acquired from the inspection value acquisition unit 224A at a predetermined ratio as the improvement target value.

The improvement inspection value inverse prediction unit 224C performs inverse prediction of what kind of inspection value should be input to the prediction model 222M as an improvement inspection value to output the improvement target value designated by the improvement target value designation unit 224B.

As shown in FIG. 7, the improvement inspection value inverse prediction unit 224C performs inverse prediction of the improvement inspection value by performing the inverse prediction process using the improvement target value designated by the improvement target value designation unit 224B. The inspection items of the improvement target value include, for example, HbA1c. The improvement inspection value includes, for example, systolic blood pressure, diastolic blood pressure, and body weight as inspection items. The prediction result of the inverse prediction process is usually not uniquely determined, and a plurality of prediction results may be obtained. Therefore, when a plurality of prediction results of the inverse prediction process are obtained, the improvement inspection value inverse prediction unit 224C selects the prediction result to be presented. In this case, the improved inspection value inverse prediction unit 224C may, for example, select a statistical mode, select a prediction result in consideration of the attribute of the inspection value that is the basis of the prediction, or randomly select a plurality of prediction results.

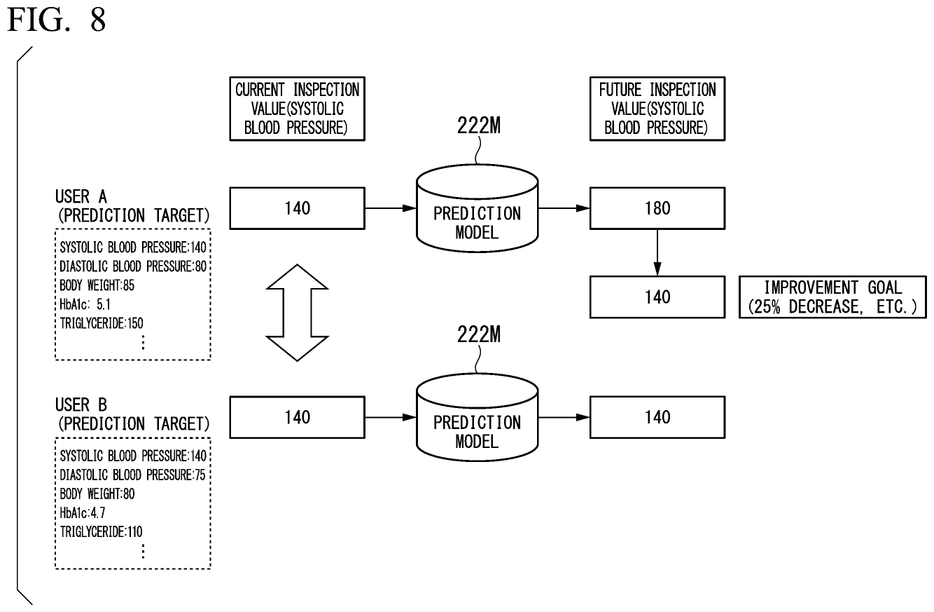

FIG. 8 is a diagram showing an example of the inverse prediction process of the improvement inspection value. In the example shown in the figure, for example, the improvement inspection value inverse prediction unit 224C refers to the future inspection value of another user having an attribute common to or similar to the prediction target user, and extracts the improvement inspection value corresponding to the improvement target value designated by the improvement target value designation unit 224B, thereby performing the inverse prediction. The inspection values of other users are, for example, accumulated future inspection values of other users predicted by the inspection value prediction unit 222B. In the example shown in the figure, the inspection item of the inspection value is “maximum hypertension”, and the current inspection value “140” of the prediction target user “user A” is input to the prediction model 222M, so that the future inspection value “180” of “user A” output from the prediction model 222M has been acquired. Further, the value obtained by reducing the future inspection value “180” of “user A” by a predetermined ratio (about 25% in the illustrated example) is acquired as the improvement target value “140”. In this example, the improvement inspection value inverse prediction unit 224C selects “user B” having a future inspection value “140” corresponding to the improvement target value “140” as a comparison target among other users having attributes common to or similar to “user A”. Then, the improvement inspection value inverse prediction unit 224C performs inverse prediction of the improvement inspection value of “user A” with reference to the current inspection value of “user B”. In this example, “user B” shows a better value than “user A” for any of the values of “minimum blood pressure”, “body weight”, “HbA1c”, and “neutral fat”, which are examples of the inspection items of the inspection values. Therefore, the improvement inspection value inverse prediction unit 224C performs the inverse prediction on the values of “minimum blood pressure”, “body weight”, “HbA1c”, and “neutral fat” for “user B” as an example of the improvement inspection values for “user A”.

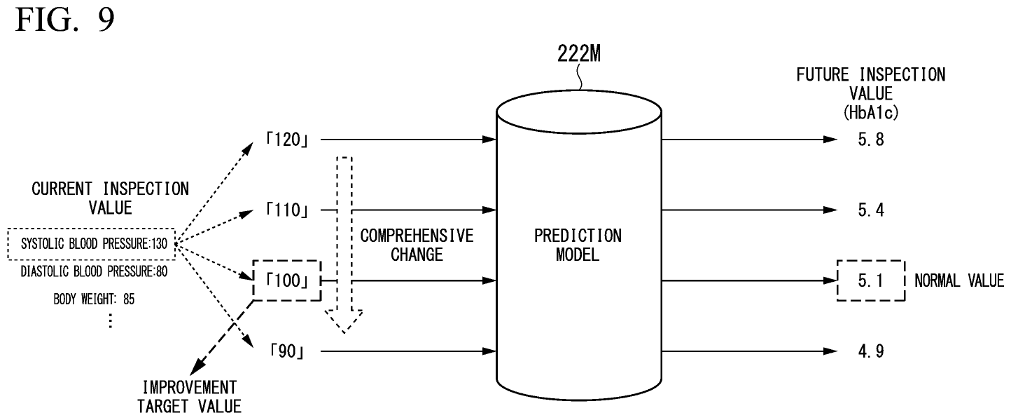

FIG. 9 is a diagram showing another example of the inverse prediction process of the improvement inspection value. In the example shown in the figure, for example, when the improvement target value is designated by the improvement target value designation unit 224B, the improvement inspection value inverse prediction unit 224C comprehensively changes the current inspection value and inputs it to the prediction model 222M, and when the inspection value output from the prediction model 222M matches the improvement target value, the inspection value input to the prediction model 222M is inversely predicted as the improvement inspection value. In the example shown in the figure, the improvement inspection value inverse prediction unit 224C outputs an instruction to the inspection value prediction unit 222B, comprehensively changes a predetermined inspection item among the inspection items of the current inspection value to be input to the prediction model 222M, and changes the future inspection value output from the prediction model 222M. In the illustrated example, the improvement inspection value inverse prediction unit 224C comprehensively changes the inspection value of “maximum blood pressure” to “120”, “110”, “100”, and “90” among the current inspection values to be input to the prediction model 222M, and comprehensively changes the inspection value of “HbA1c” output from the prediction model 222M to “5.8”, “5.4”, “5.1”, and “4.9”. Then, when the inspection value of “HbA1c” becomes “5.1” which is the normal range (for example, less than 5.2) of “HbA1c”, the improvement inspection value inverse prediction unit 224C inversely predicts the inspection value “100” of the “maximum blood pressure” input to the prediction model 222M as the improvement inspection value.

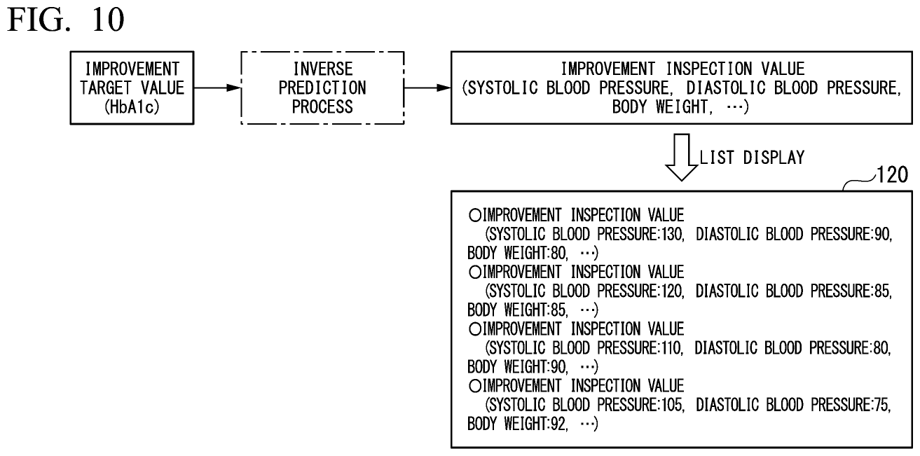

As shown in FIG. 10, when performing inverse prediction of a plurality of improvement inspection values, for example, the improvement inspection value inverse prediction unit 224C makes the display unit 130 display a list of the plurality of improvement inspection values. In this case, the improvement inspection value inverse prediction unit 224C may, for example, arrange the inspection values of a specific inspection item in ascending or descending order among the inspection items of the improvement inspection value and display the improvement inspection value in a list.

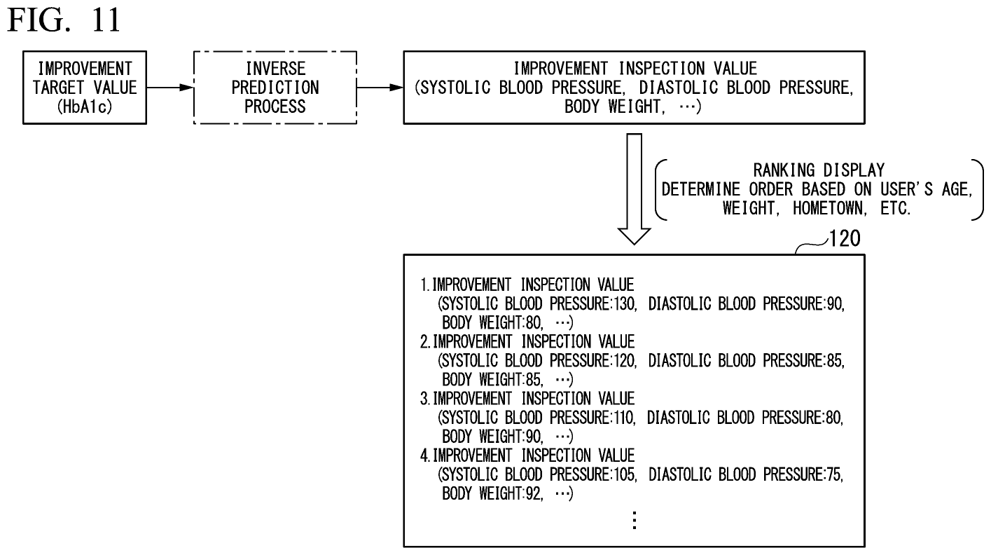

As shown in FIG. 11, when performing inverse prediction of a plurality of improvement inspection values, for example, the improvement inspection value inverse prediction unit 224C makes the display unit 130 display a plurality of improvement inspection values in a ranking. In this case, the improvement inspection value inverse prediction unit 224C may display a plurality of improvement inspection values in a ranking based on the user information of the prediction target user, for example. User information includes, for example, at least one or more of the user's age, weight, and hometown. For example, when displaying a plurality of improvement inspection values in a ranking, the higher the degree of similarity between the user information of the user who is the source of the prediction of the improvement inspection value and the user information of the prediction target user, the higher the order in which the improvement inspection value is displayed on the display unit 130 may be raised by the improvement inspection value inverse prediction unit 224C. When displaying a plurality of improvement inspection values in a ranking, the improvement inspection value inverse prediction unit 224C may extract, for example, the inspection values of the users who have the same or similar origins as the prediction target user among the users who are the sources of the improvement inspection value prediction, and may increase the order in which the improved inspection values having a high degree of agreement with the extracted inspection values are displayed on the display unit 130

The charge management unit 224D determines the charge content requested from the prediction target user based on the processing history by the inverse prediction unit 224. The charge management unit 224D registers the determined charge content in the charge management table 236 together with the processing history by the inverse prediction unit 224.

The prediction result output unit 224E outputs the improvement inspection value of the prediction target user inversely predicted by the inverse prediction unit 224 to the client device 100.

### [Processing Flow]

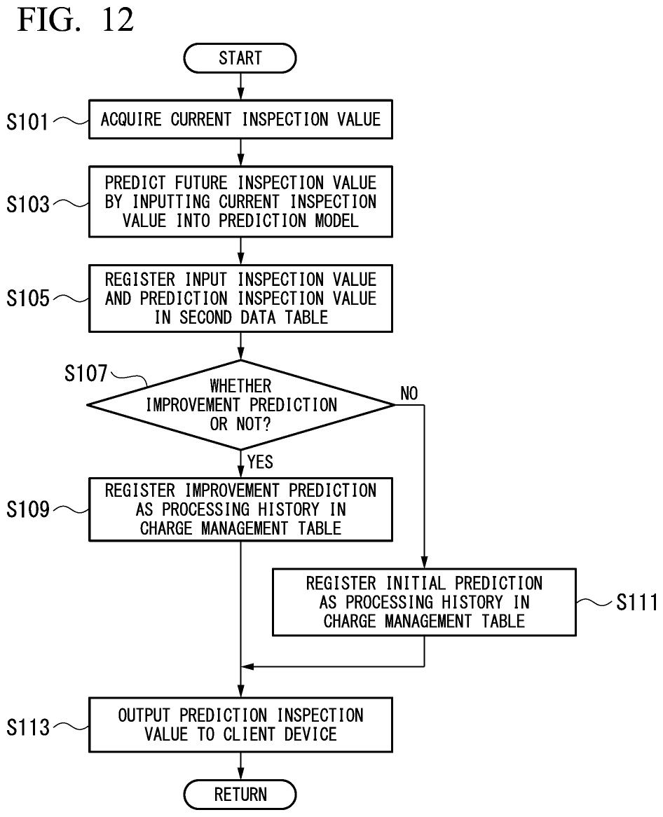

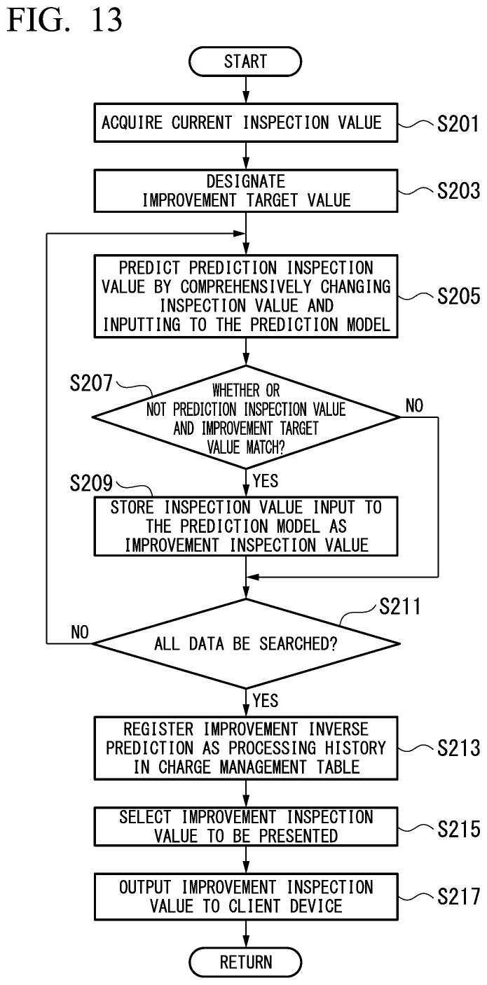

FIG. 12 is a flowchart showing an example of the inspection value prediction process. The processing of the flowchart shown in FIG. 12 is started, for example, when the server device 200 receives information requesting prediction from the client device 100.

First, the inspection value acquisition unit 222A acquires the current inspection value of the prediction target user from the client device 100 (step S101).

Next, the inspection value prediction unit 222B predicts the future inspection value of the prediction target user by inputting the current inspection value acquired by the inspection value acquisition unit 222A into the prediction model 222M (step S103).

Next, the inspection value prediction unit 222B registers the input inspection value input to the prediction model 222M and the prediction inspection value output from the prediction model 222M in the second data table 234 in association with each other (step S105).

Next, the charge management unit 222C determines whether or not the type of inspection value prediction performed by the inspection value prediction unit 222B is an improvement prediction (step S107) When the charge management unit 222C determines that the type of inspection value prediction is an improvement prediction, the charge management unit 222C registers the improvement prediction as a processing history in the charge management table 236 (step S109). On the other hand, when the charge management unit 222C determines that the type of prediction of the inspection value is not an improvement prediction, the charge management unit 222C registers the initial prediction as the processing history in the charge management table 236 (step S111).

After that, the prediction result output unit 222D outputs the prediction inspection value predicted by the inspection value prediction unit 222B to the client device 100 through the communication unit 210 (step S113).

### [Processing Flow]

FIG. 13 is a flowchart showing an example of the inspection value inverse prediction process. The processing of the flowchart shown in FIG. 13 is started, for example, when the server device 200 receives information requesting inverse prediction from the client device 100.

First, the inspection value acquisition unit 224A acquires the current inspection value of the prediction target user from the client device 100 (step S201).

Next, the improvement target value designation unit 224B designates the improvement target value based on the inspection value acquired by the inspection value acquisition unit 224A (step S203).

Next, the improved inspection value inverse prediction unit 224C outputs an instruction to the inspection value prediction unit 222B, comprehensively changes the inspection value acquired by the inspection value acquisition unit 224A, and inputs the inspection value to the prediction model 222M, thereby predicting the prediction inspection value (step S205).

Next, the improvement inspection value inverse prediction unit 224C determines whether or not the prediction inspection value predicted in step 5205 and the improvement target value designated by the improvement target value designation unit 224B match (step S207).

When determining that the prediction inspection value and the improvement target value match, the improvement inspection value inverse prediction unit 224C stores the inspection value input to the prediction model 222M in the memory as the improvement inspection value (step S209), and shifts the process to step S211. When the improvement inspection value inverse prediction unit 224C determines that the prediction inspection value and the improvement target value do not match, the process shifts to step S211 without going through the process of step S209.

Next, the improvement inspection value inverse prediction unit 224C determines whether or not all the data have been searched (step S211). When the improvement inspection value inverse prediction unit 224C determines that all the data have not been searched, the process returns to step 5205, and the processes of steps 5205 to 5211 are repeated until all the data are searched. On the other hand, when the improvement inspection value inverse prediction unit 224C determines that all the data have been searched, the process shifts to step 5213.

Next, the charge management unit 224D registers the improvement inverse prediction as the processing history in the charge management table 236 (step S213).

Next, the improvement inspection value inverse prediction unit 224C selects the improvement inspection value to be presented from the improvement inspection values stored in the memory in step 5209 (step S215).

After that, the prediction result output unit 224E outputs the improvement inspection value selected by the improvement inspection value inverse prediction unit 224C in step 5215 to the client device 100 through the communication unit 210 as the prediction result of the inverse prediction process (step S217).

### [Operation of Server Device]

Next, the operation of the server device 200 according to the first embodiment will be described.

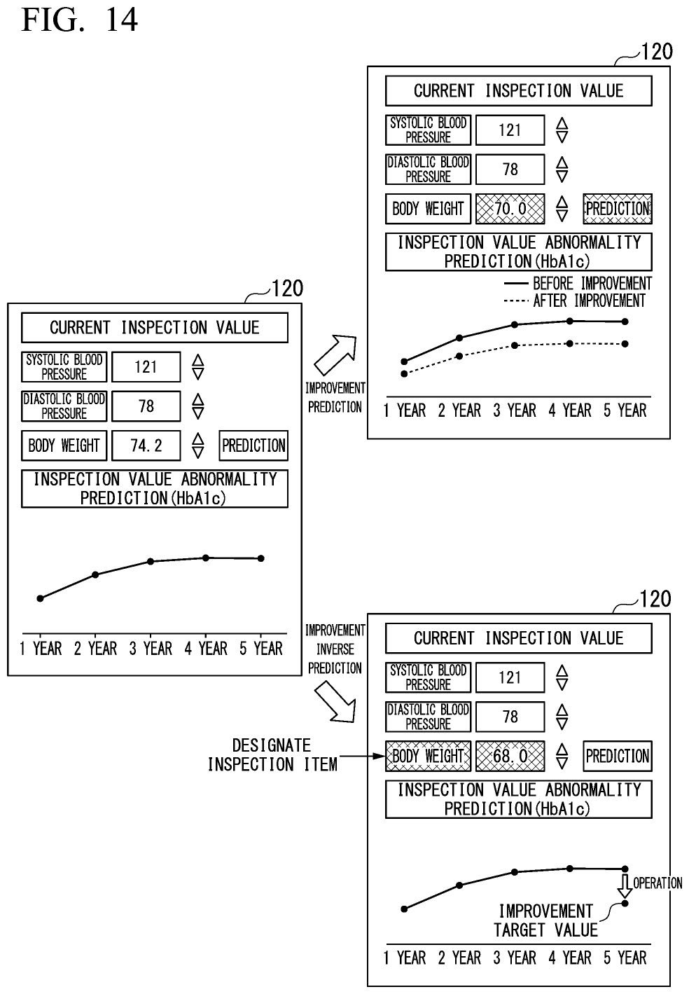

As shown in FIG. 14, when the server device 200 predicts the future inspection value by inputting the current inspection value of the prediction target user into the prediction model 222M, the server device 200 lists the current inspection value and the future inspection value on the display unit 130. In the illustrated example, the current inspection values include, for example, systolic blood pressure, diastolic blood pressure, and body weight as inspection items. Further, future inspection values include, for example, HbA1c as inspection items. In the illustrated example, future inspection values include inspection values predicted for each number of years since the current time, and time series data of these inspection values are displayed as a graph. Further, in the illustrated example, the display unit 130 is a touch panel, and displays the improvement button B1 and the prediction button B2 together with the prediction result of the current inspection value and the future inspection value of the prediction target user.

Then, when predicting the improvement of the inspection value, the server device 200 first accepts the operation of the improvement button B1 by the prediction target user. In the illustrated example, among the inspection items of the current inspection values, the parameter related to “body weight” is improved. Next, the server device 200 accepts the operation of the prediction button B2 by the prediction target user. As a result, the server device 200 displays the future inspection value predicted to be improved together with the future inspection value before the improvement on the display unit 130.

Further, when the server device 200 performs inverse prediction of improvement of the inspection value, first, the server device 200 accepts an operation for improving the inspection value in the future of the prediction target user. In the illustrated example, among the graphs showing the time-series data of future inspection values, the plot of the inspection values 5 years after the current time is dragged downward to improve the inspection values to the improvement target value. Next, the server device 200 performs inverse prediction of what kind of inspection value should be input to the prediction model 222M as an improvement inspection value to output the improvement target value. In the illustrated example, the server device 200 performs inverse prediction of the improvement inspection value after designating “body weight” as the target of improvement among the inspection items of the current inspection value.

According to the server device 200 of the first embodiment described above, the learning target user associates a plurality of inspection values acquired at the first time point with the inspection values acquired at the second time point. By inputting the inspection value of the prediction target user into the prediction model 222M learned using the learning data set including the data set, the future inspection value of the prediction target user is predicted. When the target value for the future inspection value of the prediction target user is designated, the server device 200 performs inverse prediction of what inspection value should be input to the prediction model 222M to output the designated target value. This makes it possible to provide the inversely prediction inspection value as information for improving the lifestyle of the predicted target user.

Further, the server device 200 refers to the reference data of the future inspection value of another user having an attribute common to or similar to the prediction target user, and extracts the inspection value corresponding to the designated target value to perform inverse prediction. This makes it possible for the prediction target user to perform inverse prediction of the improvement inspection value of the prediction target user by referring to the inspection value of another user who is a model for improving the inspection value.

Further, when the target value is designated, the server device 200 comprehensively changes the inspection value and inputs it to the prediction model 222M, and when the inspection value output from the prediction model 222M matches the target value, the server device 200 performs the inverse prediction based on the inspection value input to the prediction model 222M. As a result, even when the inspection value of another user who is a model for improving the inspection value cannot be obtained by the prediction target user, it is possible to performs inverse prediction of the improvement inspection value of the prediction target user.

Further, when a plurality of prediction results of inverse prediction are obtained, the server device 200 presents a plurality of prediction results to the prediction target user. This makes it possible to provide information that matches the taste of the user to be predicted.

Further, when presenting a plurality of prediction results to the prediction target user, the server device 200 raises the order of presenting the prediction result as the degree of similarity between the user information of the user who is the source of the prediction result and the user information of the prediction target user becomes higher. This makes it possible to provide information that is more consistent with the taste of the user to be predicted.

Further, the server device 200 determines the charge content requested to the prediction target user based on the processing history of the prediction unit 222 and the inverse prediction unit 224, and when the inverse prediction unit 224 performs the inverse prediction, the prediction unit 200 increases the charge required for the prediction target user as compared with the case where the improvement prediction is performed by the prediction unit 222. This makes it possible to determine the charge content required of the prediction target user by taking advantage of the usefulness of the inverse prediction process.

### Second Embodiment

Hereinafter, the second embodiment will be described. Compared with the first embodiment, the server device 200, which is an example of the inspection value prediction device according to the second embodiment, is different in that the improvement inverse prediction of the inspection value is performed using the inverse prediction model. Therefore, for the configuration and the like, the drawings and related descriptions described in the first embodiment are referred to, and detailed description thereof will be omitted.

When the improvement target value is designated, the improvement inspection value inverse prediction unit 224C uses the second inspection value used as the correct answer data (teacher data) as the learning data in the learning phase of the prediction model 222M among the data registered in the first data table 232, and performs machine learning using the first test value used as the learning data as correct answer data (teacher data), thereby learning an inverse prediction model 222MA. The inverse prediction model 222MA is an example of the “second learned model”, and the data set in which the second inspection value and the first inspection value are associated with each other is an example of the “second learned data set”. The improvement inspection value inverse prediction unit 224C performs inverse prediction of the improvement inspection value by inputting the improved value of the prediction inspection value predicted by the inspection value prediction unit 222B into the inverse prediction model 222MA as the improvement target value.

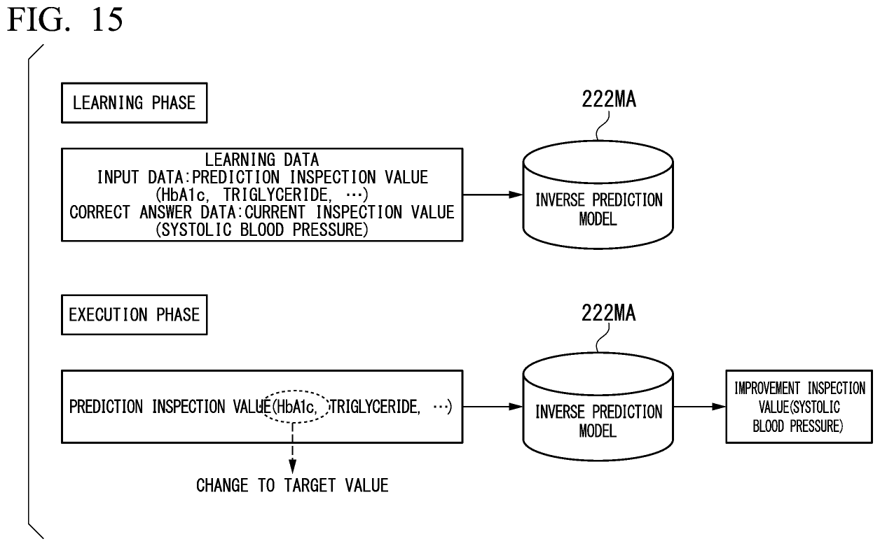

In the example shown in FIG. 15, the improved inspection value inverse prediction unit 224C uses the prediction inspection value predicted by the inspection value prediction unit 222B as learning data in the learning phase, and performs machine learning using the current inspection value acquired by the inspection value acquisition unit 224A as correct answer data (teacher data), thereby learning the inverse prediction model 222MA. Predictive inspection values include, for example, HbA1c and triglycerides as inspection items. Current inspection values include, for example, systolic blood pressure as inspection items.

The improvement inspection value inverse prediction unit 224C performs inverse prediction of the improvement inspection value by inputting the prediction inspection value predicted by the inspection value prediction unit 222B into the inverse prediction model 222MA in the execution phase. Predictive inspection values include, for example, HbA1c and triglycerides as inspection items. The improvement inspection value includes, for example, systolic blood pressure as an inspection item.

### [Processing Flow]

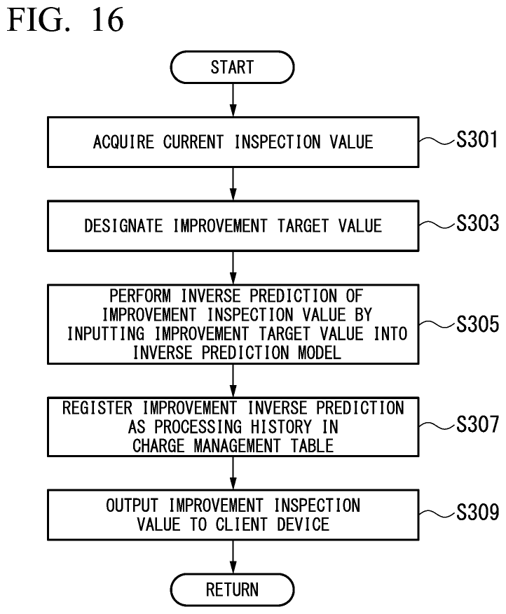

FIG. 16 is a flowchart showing an example of the inspection value inverse prediction process. The processing of the flowchart shown in FIG. 16 is started, for example, when the server device 200 receives a request for inverse prediction from the client device 100.

First, the inspection value acquisition unit 224A acquires the current inspection value of the prediction target user from the client device 100 (step S301).

Next, the improvement target value designation unit 224B designates the improvement target value based on the inspection value acquired by the inspection value acquisition unit 224A (step S303).

Next, the improvement inspection value inverse prediction unit 224C performs inverse prediction of the improvement inspection value by inputting the improvement target value designated by the improvement target value designation unit 224B into the inverse prediction model 222MA (step S305).

Next, the charge management unit 224D registers the improvement inverse prediction as the processing history in the charge management table 236 (step S307).

After that, the prediction result output unit 224E outputs the improvement inspection value inversely predicted by the improvement inspection value inverse prediction unit 224C to the client device 100 through the communication unit 210 as the prediction result (step S309).

As described above, according to the server device 200 of the second embodiment, when the improvement target value is designated, the inverse prediction model 222MA is learned using the second learning data set in which the input and output of a plurality of data sets in which the first inspection value and the second inspection value are associated with each other are replaced with the first learning data set. The improvement inspection value inverse prediction unit 224C performs inverse prediction of the improvement inspection value by inputting the improved value of the prediction inspection value predicted by the inspection value prediction unit 222B into the inverse prediction model 222MA as the improvement target value. As a result, even if the inspection value of another user who is a model for improving the inspection value cannot be obtained by the prediction target user, it is possible to inversely predict the improvement inspection value of the prediction target user while suppressing the processing load.

The inspection value prediction function in the above-described embodiment may be realized by the client device 100. Further, the inspection including the inspection value prediction function in the above-described embodiment may be realized in a value prediction system that includes an inspection value prediction device composed of the server device 200 or the client device 100, and an application program that operates in the terminal device that communicates with the inspection value prediction device.

Although some embodiments of the present invention have been described, these embodiments are presented as examples and are not intended to limit the scope of the invention. These embodiments can be implemented in variations thereof, and various omissions, replacements, and changes can be made without departing from the gist of the invention. These embodiments and variations thereof are included in the scope of the invention described in the claims and the equivalent scope thereof, and are included in the scope and gist of the invention.

The present invention can be widely applied to an inspection value prediction device, an inspection value prediction system, and an inspection value prediction method, and makes it possible to provide information for improving a user's lifestyle.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications that would fall within the scope and spirit of the inventions.

What is claimed is:

1. An inspection value prediction device comprising:
a prediction unit configured to input an inspection value of a user to a first learned model, which has been learned using a first learning data set containing series data of a plurality of inspection values of the user, to predict a future inspection value of the user; and
an inverse prediction unit configured, when a target value pertaining to the future inspection value of the user is designated, to perform an inverse prediction of the inspection value that causes the designated target value to be output when the inspection value is input to the first learned model.

2. The inspection value prediction device according to claim 1, wherein the inverse prediction unit is configured to perform the inverse prediction by referring to reference data of the future inspection value of other users having attributes common to or similar to the user, and extracting the inspection value corresponding to the designated target value.

3. The inspection value prediction device according to claim 2, wherein the reference data is an accumulation of results predicted by the prediction unit.

4. The inspection value prediction device according to claim 1, wherein, when the target value is designated, the inverse prediction unit comprehensively changes the inspection value and inputs it to the first learned model, and performs the inverse prediction based on the inspection value input to the first learned model when the inspection value output from the first learned model coincides with the target value.

5. The inspection value prediction device according to claim 1, wherein, when the target value is designated, the inverse prediction unit performs the inverse prediction by inputting the designated target value to a second learned model, which has been learned using a second learning data set in which input and output of the series data of the plurality of inspection values are replaced with the first learning data set.

6. The inspection value prediction device according to claim 1, wherein, when a plurality of prediction results of the inverse prediction are obtained, the inverse prediction unit presents the plurality of prediction results to the user.

7. The inspection value prediction device according to claim 6, wherein, when presenting the plurality of prediction results to the user, the inverse prediction unit raises an order of presenting the prediction result as a degree of similarity between a user information of the user who is a source of the prediction result and the user information of the user becomes higher.

8. The inspection value prediction device according to claim 7, wherein the user information includes at least one or more of age, weight, and place of origin of the user.

9. The inspection value prediction device according to claim 1, further comprising:
a charge management unit configured to determine a charge content requested to the user based on a processing history of the prediction unit and the inverse prediction unit, the charge management unit increasing a charge required of the user when processing by the inverse prediction unit is performed as compared with a case where a processing by the prediction unit is performed.

10. An inspection value prediction system comprising:
an inspection value prediction device including
a prediction unit configured to input an inspection value of a user to a first learned model, which has been learned using a first learning data set containing series data of a plurality of inspection values of the user, to predict a future inspection value of the user, and
an inverse prediction unit configured, when a target value pertaining to the future inspection value of the user is designated, to perform an inverse prediction of the inspection value that causes the designated target value to be output when the inspection value is input to the first learned model; and
an application program that operates in a terminal device that communicates with the inspection value prediction device,
wherein the application program causes the future inspection value of the user predicted by the inspection value prediction device to be displayed on the terminal device, and
receives input of a target value regarding the future inspection value of the user and transmits it to the inspection value prediction device.

11. An inspection value prediction method comprising:
predicting a future inspection value of the user by inputting an inspection value of a user to a first learned model, which has been learned using a first learning data set containing series data of a plurality of inspection values of the user; and
when a target value pertaining to the future inspection value of the user is designated, performing an inverse prediction of the inspection value that causes the designated target value to be output when the inspection value is input to the first learned model
